(19) United States
(12) Patent Application Publication
(10) Pub. No.: US 2024/0084157 A1
(43) Pub. Date: Mar. 14, 2024

(54) METHODS OF COATING POLYMERS AND REDUCTION IN PROTEIN AGGREGATION

(71) Applicant: Glycome Biopharma Limited, Co Cork (IE)

(72) Inventors: Eoin Scanlan, Dublin (IE); Graham Smith, Co Cork (IE); Silvia Fogli, Co Cork (IE); Anna Testolin, Co Cork (IE); Paula Colavita, Dublin (IE)

(73) Assignee: Glycome Biopharma Limited, Co Cork (IE)

(21) Appl. No.: 18/270,626

(22) PCT Filed: Jan. 4, 2021
(86) PCT No.: PCT/EP2021/050029

§ 371 Date: Jun. 30, 2023

(51) Int. Cl. C09D 105/08; A61M 5/31; C08J 7/04; C09D 103/02; C09D 105/00; C09D 105/02

(52) U.S. Cl. CPC C09D 105/08; A61M 5/31; C08J 7/0427; C09D 103/02; C09D 105/00; C09D 105/02; C08J 2345/00; C08J 2403/02; C08J 2405/00; C08J 2405/02; C08J 2405/08

(57) ABSTRACT

A method of coating a polymer surface, the method comprising: providing a polymer having a surface, optionally, treating at least a portion of the polymer surface with an oxidising agent, treating at least a portion of the polymer surface with a composition comprising a polysaccharide, oligosaccharide, polyol or mixture thereof, and incubating the treated polymer with the composition for a predetermined time. Also disclosed are polymers comprising such a coating, vessels comprising such coated polymers and medicals devices comprising such polymers.

### FIELD OF THE INVENTION

The present invention relates to methods of coating polymer surfaces, to coated polymers obtained thereby, and to methods of reducing protein aggregation on a polymer surface. The present invention also relates to vessels for fluids, medical devices and syringes comprising coated polymers.

### BACKGROUND OF INVENTION

Protein aggregation and denaturing in formulations of proteinaceous compositions contained in devices may occur and this causes problems in diagnostics, analysis and drug delivery. Control of protein aggregate formation and denaturing is problematic.

Non-specific protein adsorption is a complex event. The process is governed by the properties of the protein (e.g. structure, size, and distribution of charge and polarity), the properties of the material surface (e.g. charge, roughness, and state of surface energy) environmental conditions (e.g. pH, ionic strength and temperature) and the kinetics of the adsorption process.

Proteins may bind non-specifically at the surface of materials used during sample preparation, such as pipette tips, sample tubes, well plates and vials, which can result in loss of experimental accuracy. Regulatory guidelines require bioanalytical methods to be validated not only in terms of linearity, sensitivity, accuracy, precision, selectivity and stability, but also in terms of carryover. Carryover results from the nonspecific adsorption of analyte(s) to parts of the analytical system and thus introduces bias in both identification and quantification assays. Hence, linearity, sensitivity and repeatability of the analyses are negatively affected.

Disposable systems have gained increased acceptance for large scale storage during manufacturing and processing of recombinant proteins and monoclonal antibodies in liquid and frozen forms. Interactions between containers and pharmaceutical solutions is important: the physicochemical properties of container materials contribute toward maintaining the integrity and stability of drug substances. Adsorption of a protein on to a container surface may result in loss of protein potency within a solution arising from changes in concentration, protein denaturation and/or degradation. Protein aggregation and denaturing of pharmaceutical compositions (such as antibodies, proteins and other peptides, for example, erythropoietin, interferon-gamma, infliximab, etanercept, and adalimumab, all of which may be delivered in pre-filled in syringes) may also cause adverse immune response and has resulted in the withdrawal of some biopharmaceuticals from the market.

Surface modifications of the materials used to produce medical devices and vessels for delivery of compositions is one approach to attempt to mitigate the problem. Surface modifications of protein contacting materials used in manufacturing storage, e.g. ethylene vinyl acetate (EVA) copolymers and low-density polyethylene (LDPE), can potentially mitigate aggregate formation and protein adsorption thereby offering improved product quality and safety. Materials include glass or polymers (e.g. cyclic olefin polymers, COPs) that may be modified by applications of an inorganic coating on the surface that will be in contact with the composition.

WO-A-2020/092373 discloses a drug container having a thermoplastic wall, a PECVD (plasma-enhanced chemical vapor deposition) drug-contact coating, and a polypeptide composition contained in the lumen. The drug-contact coating is on or adjacent to the internal surface of the container, positioned to contact a fluid in the lumen, and consists essentially of SiOₓC_{y}H_{z}, a barrier to reduce corrosion.

US-A-2015/0126941 discloses a filled package comprising a vessel, a barrier coating a protective coating on the vessel, and a fluid composition contained in the vessel in order to increase the shelf life of the package. The barrier coating is of SiOₓ (x is 1.5 to 2.9). The protective coating comprises a layer of a saccharide to stop leaching.

There is a need to provide surfaces of materials that are less prone to protein aggregation and denaturing and do not suffer from the problems of the prior art.

It is an aim of the present invention to address this need.

### SUMMARY OF INVENTION

The present invention accordingly provides in a first aspect a method of coating a polymer surface, the method comprising: a) providing a polymer having a surface, b) optionally, treating at least a portion of the polymer surface with an oxidising agent, c) treating at least a portion of the polymer surface with a composition comprising a polysaccharide, oligosaccharide, polyol or mixture thereof, and d) incubating the treated polymer with the composition for a predetermined time.

Surprisingly, use of a polysaccharide, oligosaccharide, polyol or mixture thereof as a coating significantly reduces protein adsorption and/or aggregation and may also reduce oligonucleotide adsorption and/or aggregation.

The composition may be applied above one or more other coating layers (except a layer of silica) already deposited on the polymer surface. Preferably, the polymer surface does not comprise a silica coating.

However, preferably the composition is applied directly to the polymer surface, needing no inorganic layers already deposited on the polymer surface. Thus, preferably, the method comprises treating the polymer surface directly. Generally, any suitable polymer (e.g. EVA, polyolefin (for example polyethylene or polypropylene), a polyester (for example polyethylene terephthalate), a polycarbonate, or any combination or copolymer of any of these) may be used in the method, but preferably the polymer may comprise a cyclic olefin polymer or co-polymer. The polymer (for example the cyclic olefin polymer) may comprise, at least partially, recycled polymer.

Cyclic olefin polymers are useful as high temperature polymers with outstanding optical properties, good chemical and heat resistance, and excellent dimensional stability. The COP may be produced from cyclic olefin monomers such as norbornene, cyclopentadiene (CPD), and/or dicyclopentadiene (DCPD).

Although, it is thought that a number of polysaccharides, oligosaccharides, polyols or mixtures thereof may be useful in the method, the polysaccharide etc. may comprise a hexose derived polysaccharide. The polysaccharides may be polyhydroxylated. Generally, the polysaccharides may provide a relatively hydrophilic surface (e.g. water contact angle below 80°, below 70°, below 60°, below 50°, or lower), preferably once applied to the polymer surface.

The preferred polysaccharide is selected from dextran, cellulose, one or more polyols, dextrin, polygalacturonic acid, hyaluronic acid, or a combination of two or more of these polysaccharides.

Use of these polysaccharides, oligosaccharides, polyols or mixtures thereof is greatly advantageous because the inventors have determined they significantly reduce protein aggregation when applied to COP surfaces.

The oxidising agent preferably affects the surface of the polymer but preferably does not adversely affect the bulk of the polymer. The oxidising agent may comprise a peroxide, optionally may comprise hydrogen peroxide, optionally may comprise hydrogen peroxide in 30% w/w aqueous solution. Generally, peroxide and/or other oxidising agents may also be suitable, for example O₃, ozonated water, H₂O₂ with and without decomposition catalysts (e.g. Cu ions, Fe ions, manganese oxide), periodate, hypochlorite, and/or permanganate.

The predetermined time may be in the range 0.5 mins to 240 mins. Other optional ranges for the predetermined time may be 1 min to 120 min, 1 min to 60 min, 1 min to 30 min, 1 min to 20 min, or 1 min to 10 min.

Treating at least a portion of the polymer surface and/or incubation may be at a temperature in the range 10° C. to 90° C., optionally 10° C. to 70° C.

Treating at least a portion of the polymer surface and/or treatment during incubation may comprise mechanical, chemical or electromagnetic acceleration of the process e.g. by sonication, microwave irradiation, and/or ion-catalysis.

The composition may be in aqueous solution. Thus, the composition may comprise water. One or more co-solvent(s) may also be present, if suitable.

In some embodiments, the composition may comprise an oxidising agent. The oxidising agent in the composition may comprise a peroxide, optionally may comprise hydrogen peroxide, optionally may comprise hydrogen peroxide in 30% w/w aqueous solution.

The polymers obtained by the present method have significantly reduced protein aggregation.

The present invention accordingly provides in a second aspect a coated polymer obtainable by coating at least one surface of a polymer according to a method of the first aspect.

Optionally, the coated polymer does not comprise a silica coating.

The present invention accordingly provides in a third aspect a polymer having a coating on at least one surface, the coating comprising a polysaccharide directly contacting the surface of the polymer.

The polymer preferably comprises a cyclic olefin polymer.

The polysaccharide preferably comprises dextran, cellulose, polyols (e.g. hydrogenated hydrolysates, e.g. of starch), dextrin, polygalacturonic acid, hyaluronic acid, or a combination of two or more of these polysaccharides.

Coated polymers of the present invention have a further great advantage in that they enhance the thermal and intrinsic stability of compositions stored in contact with the coated surface (e.g. when compared with the uncoated surface or other materials).

Thus, the present invention provides in a fourth aspect, use of a vessel comprising a coated polymer according to the third aspect to store a pharmaceutical composition (optionally a peptide composition), thereby enhancing the intrinsic and/or thermal stability of the pharmaceutical composition.

The present invention accordingly provides in a fifth aspect a method of reducing protein or oligonucleotide aggregation or adsorption on a polymer surface, the method comprising: a) providing a polymer as discussed above and according to the second aspect, and b) contacting the surface with a proteinaceous or oligonucleotide composition. As discussed above, this is advantageous because it provides for improved storage conditions e.g. allowing storage at higher temperature and/or for longer than previously.

The proteinaceous composition may comprise a pharmaceutical proteinaceous composition. The pharmaceutical proteinaceous composition may comprise a monoclonal antibody composition, or a peptide hormone.

In some embodiments, the pharmaceutical proteinaceous composition may comprise one or more of a vaccine (e.g. a vaccine comprising a peptide), erythropoietin, interferon (α-,β-, and/or γ-interferon), infliximab, etanercept, adalimumab, rituximab, infliximab, trastuzumab, insulin, glucagon, and/or a gonadotrophin.

The pharmaceutical composition may comprise an injectable composition. Examples of injectable compositions may include:
Abarelix-Depot (hormone);
AbobotulinumtoxinA Injection (Dysport);
Acetadote (Acetylcysteine Injection);
Actemra (Tocilizumab Injection);
Acthrel (Corticorelin Ovine Triflutate for Injection);
Actimmune (Interferon gamma-1b);
Adacel (vaccine);
Adalimumab (Humira);
Adenoscan (Adenosine Injection);
Aldurazyme (Laronidase);
Alglucerase Injection (Ceredase);
Alkeran Injection (Melphalan Hcl Injection);
ALTU-238 (human growth hormone);
Arzerra (Ofatumumab Injection);
Avastin (Bevacizumab);
Azactam Injection (Aztreonam Injection);
BayHepB (hepatitis b immune globulin human); antibody);
BayTet (Tetanus Immune Globulin (Human)); antibody);
Bexxar (Tositumomab) (antibody);
Blenoxane (Bleomycin Sulfate Injection; peptide antibiotic);
Botox Cosmetic (OnabotulinumtoxinA for Injection; protein);
BR3-FC (protein);
Briobacept (antibody);
BTT-1023 (antibody);
Byetta (Exenatide; peptide);
Campath (Altemtuzumab; antibody)
Canakinumab Injection (Ilaris; antibody)
Carticel; (chondrocytes cells)
Cathflo; (Alteplase; protein)
Cerezyme (Imiglucerase) (enzyme);
Certolizumab Pegol (Cimzia; antibody);
Choriogonadotropin Alfa, recombinant (r-hCG) for Injection (Ovidrel; peptide hormone);
Chorionic gonadotropin (hCG) for Injection (Pregnyl; Follutein; Profasi; Novarel; peptide hormone);
Clofarabine Injection (Clolar, Evoltra; purine nucleoside);
Colistimethate Injection (Coly-Mycin M); (polypeptide)
Corifollitropin alfa (Elonva; peptide hormone);
Copaxone (Glatiramer Acetate; mix of peptides);
Cubicin (Daptomycin Injection; cyclic lipopeptide);
Dacetuzumab (antibody);
Darbepoietin Alfa (protein);
DDAVP Injection (Desmopressin Acetate Injection peptide hormone);
Denosumab Injection (Prolia; antibody);
DMOAD (Disease-Modifying OsteoArthritis Drugs; class of compounds some of which are peptides);
Ecallantide Injection (Kalbitor; protein);
Engerix (vaccine);
Enbrel (etanercept; protein);
Epratuzumab (antibody);
Erbitux (Cetuximab; antibody);
Erythropoietin (peptide hormone);
Essential Amino Acid Injection (Nephramine) (mix of amino acids);
Fabrazyme (Agalsidase beta; enzyme);
Fluarix Quadrivalent (vaccine);
Fludara (Fludarabine Phosphate); (nucleotide analog derivative);
Follitropin Alfa Injection (Gonal-f RFF; Cinnal-f; Fertilex; Ovaleap; Bemfola; peptide hormone);
Follitropin Beta Injection (Follistim; Follistim AQ Cartridge; Puregon; peptide hormone);
Follitropin Delta Injection (Rekovelle; peptide hormone);
Forteo (Teriparatide (rDNA origin) Injection; peptide hormone);
Foscamet Sodium Injection (Foscavir);
Fuzeon (enfuvirtide; peptide);
GA101 (Obinutuzumab; antibody);
Ganirelix (Ganirelix Acetate Injection; peptide);
Gardasil (vaccine);
GC1008 (Fresolimumab; antibody);
Gemtuzumab Ozogamicin for Injection (Mylotarg); (antibody-drug conjugate)
Golimumab Injection (Simponi Injection; antibody);
GlucaGen (Glucagon; peptide hormone);
Havrix; (vaccine)
Herceptin (Trastuzumab; antibody);
hG-CSF (Human granulocyte colony-stimulating factor; protein);
Humalog (Insulin lispro; peptide hormone);
Human Growth Hormone;
Humegon (Human gonadotropin; peptide hormone);
Humulin (Insulin and analogues (modified form of insulin?), peptide hormone);
IncobotulinumtoxinA for Injection (Xeomin; protein)
Increlex (Mecasermin [rDNA origin] Injection); (human growth factor)
Infanrix; (vaccine)
Insulin (peptide hormone);
InsulinAspart [rDNA origin] Inj (NovoLog); (peptide hormone)
Insulin Glargine [rDNA origin] Injection (Lantus); (peptide hormone)
Insulin Glulisine [rDNA origin] Inj (Apidra); (peptide hormone)
Interferon alfa-2b, Recombinant for Injection (Intron A); (protein)
Interferon beta-1b, Recombinant for Injection (Betaferon; protein)
Iplex (Mecasermin Rinfabate [rDNA origin] Injec-tion); (human growth factor)
Iprivask (Desirudin for injection; protein);
Istodax (Romidepsin for Injection); (peptide)
Kepivance (Palifermin; keratinocyte growth factor);
Keratinocyte (epidermal cells);
KFG (keratinocyte growth factor);
Kineret (Anakinra; protein);
Kinlytic (Urokinase Injection; enzyme)
Kinrix; (vaccine)
Lente (L); (Insulin zinc; peptide hormone)
Leptin; (peptide hormone)
Levemir; (insulin analogue; peptide hormone)
Leukine (Sargramostim; protein)
Leuprolide Acetate injection (Lupron; peptide);
Levothyroxine (amino acid);
Lexiscan (Regadenoson Injection) (nucleoside);
Liraglutide injection (Victoza; peptide);
Lucentis (Ranibizumab Injection) (antibody);
Lumizyme; (Alglucosidase alfa; enzyme);
Lutropin alfa (LH) for injection (Luveris; peptide hormone);
Menactra (vaccine);
Menotropins for Injection (Menopur; Repronex; Pergonal; peptide hormones);
MetMab (Onartuzumab; antibody);
Miacalcin; (polypepide);
Mipomersen (Kynamro oligonucleotide);
Myozyme (Alglucosidase alfa) (enzyme);
NEO-GAA; (Avalglucosidase alfa enzyme);
Neupogen (Filgrastim; protein);
Novolin; (Novolin R: Insulin; Novolin N: Insulin isophane; peptide hormone);
NeoRecormon (Epoetin beta; protein);
NPH (N) (Humulin N; Novolin N; Isophane Insulin; peptide hormone);
Novolin 70/30 Innolet (70% NPH, Human Insulin Isophane Suspension and 30% Regular, Human Insulin Injection); (peptide hormone);
Nplate (Romiplostim; protein);
Octreotide Acetate Injection (Sandostatin LAR; peptide);
Ocrelizumab (Ocrevus; antibody);
Orencia (Abatacept; antibody);
Osteoprotegrin (protein);
Oxytocin Injection (Pitocin; peptide hormone);
Panitumumab Injection for Intravenous Use (Vectibix; antibody);
Parathyroid Hormone; (peptide hormone);
Pediarix (vaccine);
Peginterferon (Peginterferon alfa-2a: Pegasys; Peginterferon alfa-2b: PEGintron; Sylatron);
Pegfilgrastim (Neulasta; Ristempa; protein);
Pegfilgrastim-cbqv (Udenyca; protein);
Pertuzumab (2C4; Omnitarg; Perjeta; antibody);
Pramlintide Acetate Injection (Symlin; Symlin pen (device for administration); peptide hormone);
R-Gene 10 (Arginine Hydrochloride Injection) (amino acid);
Raptiva (Efalizumab; antibody);
Recombivarix HB (vaccine);
Remicade (Infliximab; antibody);
Retrovir IV (Zidovudine Injection) (nucleoside);
rhApo2L/TRAIL (Dulanermin; protein);
Rituximab (MabThera; Rituxan; Truxima; antibody);
Roferon-A (Interferon alfa-2a; protein);
Somatropin for injection (Accretropin; Genotropin; Humatrope; Saizen; Norditropin; Valtropin);
Somatropin (rDNA origin) for Injection (Nutropin; Nutropin Depot; Nutropin AQ; Serostim LQ; Onmitrope; Tev-Tropin);
Stelara Injection (Ustekinumab; antibody);
Stemgen (Ancestim; protein);
Telavancin for Injection (Vibativ; lipoglycopeptide);
Tenecteplase (Metalyse; TNKase; protein);
Thymoglobulin (Anti-Thymocyte Globulin (Rabbit); antibody);
Thyrogen (Thyrotropin Alfa for Injection; peptide hormone);
Trastuzumab-Dm1 (antibody-drug conjugate);
Travasol (Amino Acids (Injection));
Trelstar (Triptorelin Pamoate for Injectable Suspension; peptide);
Twinrix (vaccine);
Typhoid Vi-Polysaccharide Vaccine (Thyphim Vi; vaccine);
Urofollitropin for Injection (Bravelle; Fertinex; Fertinorm; Metrodin; peptide hormone);
Ultralente (U) (Extended Insulin Zinc; peptide hormone);
Vancomycin Hydrochloride (Vancomycin Hydrochloride Injection; glycopeptide);
VAQTA (vaccine);
Xolair (Omalizumab; antibody);
Zenapax (Daclizumab; antibody); and/or
Zevalin (Ibritumomab tiuxetan; antibody).

The present invention according provides in a sixth aspect a vessel for fluids comprising a polymer as discussed above and as discussed in the second aspect.

The vessel may be selected from a multi-well plate, a pipette, a bottle, a flask, a vial, an Eppendorf tube, and/or a culture plate.

The present invention is particularly useful for medical devices. Thus, the present invention accordingly provides in a seventh aspect a medical device comprising a polymer as discussed above and in the second aspect.

The medical device may be a dispensing tube, a channel and/or a syringe, for example a disposable syringe.

In this specification, and unless the context suggests otherwise, cyclic olefin polymers (COP) as referred to herein include cyclic olefin copolymers (COC). Proteinaceous compositions as referred to herein include peptides, oligopeptides, and/or polypeptides in a composition and may include additional components such as excipients (e.g. sugar compounds such as lactose, dextrin, glucose, sucrose, and/or sorbitol), salts, solvent (and/or co-solvents) and other non-proteinaceous active pharmaceutical components, and their formulations. Polysaccharide includes oligosaccharides, polyols or mixtures thereof.

### BRIEF DESCRIPTION OF FIGURES

Embodiments of the present invention will be described in more detail with reference to the accompanying Figures in which:

FIG. 1. (a) Quantitative determinations of adsorbed BSA-FITC at pristine TOPAS™ (TW) and ZEONOR™ (ZW) surfaces retained in the form of a hard layer (black bars) and a soft layer (grey bars). (b) Rinsing protocols developed to tailor assay sensitivity to hard layer (HL) and soft layer (SL).

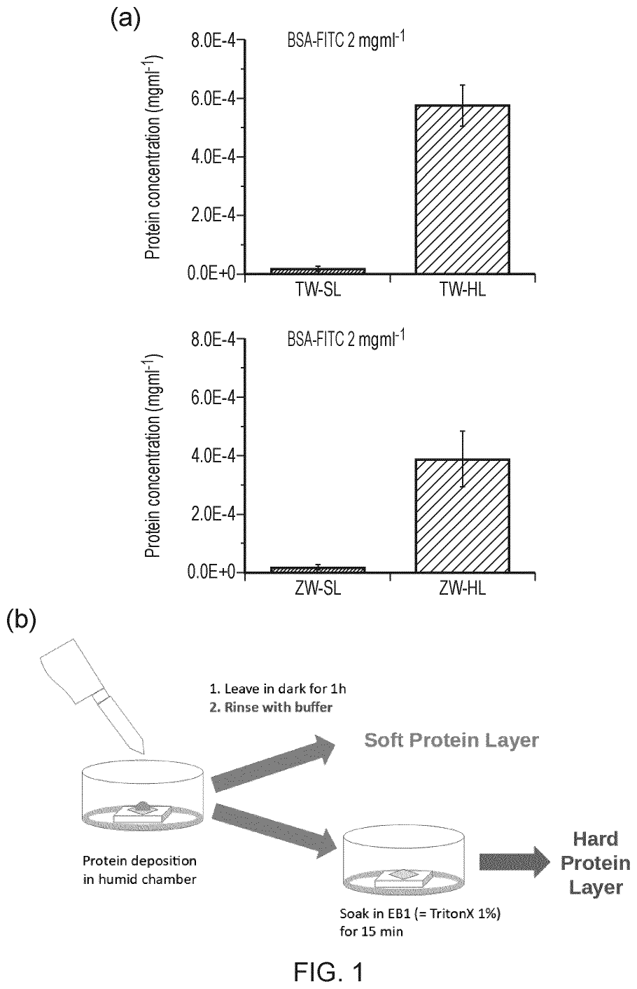

FIG. 2. Summary of protein surface coverage determined at pristine and treated surfaces resulting from 2 mg mL⁻¹ BSA-FITC incubation experiments at COP surfaces.

FIG. 3. Comparison of emission data (ΔMFI) resulting from 2 mg mL⁻¹ BSA-FITC incubation experiments at COP surfaces obtained via microscopy. The pristine surface is used as reference 100% emission.

FIG. 4. Comparison of emission data (ΔMFI) resulting from 2 mg mL⁻¹ BSA-FITC incubation experiments at COP surfaces obtained via microscopy. The pristine surface is used as reference 100% emission.

FIG. 5. Summary of protein surface coverage determined at pristine and PGA-treated syringes resulting from 2 mg mL⁻¹ BSA-FITC incubation experiments.

FIG. 6. Summary of protein surface coverage determined at pristine and PGA-treated syringes resulting from 2 mg mL⁻¹ Insulin-FITC incubation experiments.

FIG. 7. (a) GATR-FTIR spectra of a Zeonor™® coupon surface after rinsing with water (ZW) and after treatment in H₂O₂ at 50° C. for 30 min (ZP50). (b) UV-Vis absorbance spectra of a 1 mm Zeonor™® coupon after rinsing with water only (ZW) and after treatment with H₂O₂ at 50° C. for 30 min (ZP50).

FIG. 8. (a) GATR-FTIR spectra of a Zeonor™® coupon surface after rinsing with water (ZW) and after oxidising treatment via exposure to a UV/ozone lamp for 5 (ZU5) and 10 min (ZU10). (b) UV-Vis absorbance spectra of a 1 mm Zeonor™® coupon after rinsing with water only (ZW), and after and after oxidising treatment via exposure to a UV/ozone lamp for 5 (ZU5) and 10 min (ZU10).

FIG. 9. Water contact angle measurement obtained at COP coupon surfaces after rinsing in water and undergoing a range of treatment conditions with and without PGA.

FIG. 10. Comparison between the surface composition of a coupon of TOPAS and syringe type S1, analysed by FTIR.

FIG. 11. Comparison between the surface composition of a coupon of Zeonor and syringe type S3, analysed by FTIR.

FIG. 12. Comparison between the surface composition of a coupon of Zeonex and a syringe type S3, analysed by FTIR.

FIG. 13. Comparison between the surface composition of a coupon of TOPAS and a syringe type S2, analysed by FTIR.

FIG. 14. Comparison between the surface composition of a coupon of Zeonor and syringe type S2, analysed by FTIR.

FIG. 15. Comparison between the surface composition of a coupon of Zeonex and syringe type S2, analysed by FTIR.

### DETAILED DESCRIPTION

The studies herein use a fluorescently labelled globular protein, BSA-FITC to monitor the extent of protein surface adsorption at cyclo-olefin polymers (COP) materials. BSA is typically used as an indicator of the ability of a surface to resist unspecific protein adsorption. A second (fluorescently labelled) protein, Insulin-FITC, has been used to confirm the generality of the effect and its applicability to a therapeutic protein.

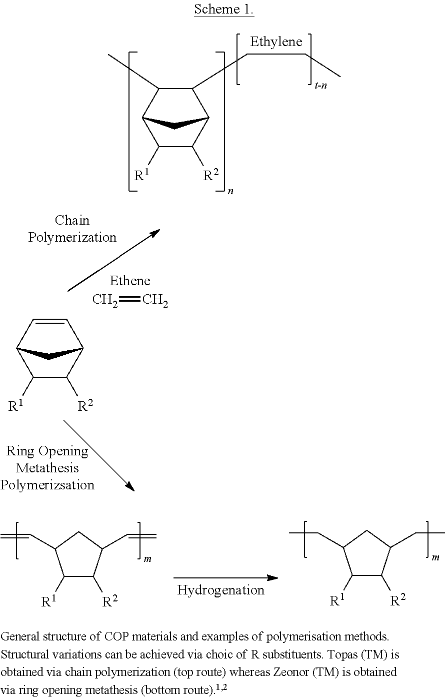

Three types of COP materials were investigated: TOPAS® (T) (Topas™ Advanced Polymer), ZEONOR® (Z) and ZEONEX® (Zeon Corporation) sourced from commercial suppliers in 1 mm thick coupon form. These materials are used by biodevice manufacturers for the biopharmaceutical industry. Scheme 1 shows a general structure of COP materials of different kinds; structural variations can be achieved via changes in the substituent groups which provide tunable properties. ¹ Shin J. Y. et al., Pure and Applied Chemistry, (2005) 77: 801-814.² Nunes et al. Microfluid Nanofluid (2010) 9:145-161.

To verify that the results of coupons were generalisable to biomedical devices, studies were conducted using selected syringe biodevices sold for pre-filled biotherapeutics sourced from three different manufacturers: Manufacturers #1-#3. All the syringes are of COP materials, while those manufactured by Manufacturer #1 are siliconized in their internal surface (barrel).

The adsorption of proteins to surfaces is a complex process; proteins typically undergo complete and/or partial denaturation when adsorbed at surfaces and the strength and nature of the interactions involved in protein adhesion varies.

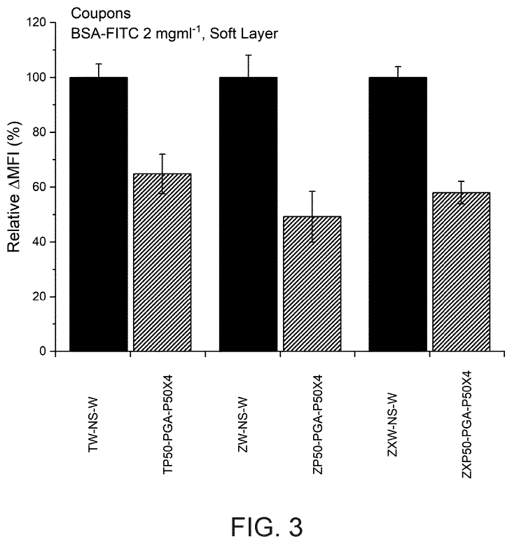

FIG. 3a shows quantitative determinations of the amount of BSA-FITC adsorbed at coupons of pristine Topas™ and Zeonor™.

Coupons (1.25 cm²) of these two COP materials were immersed in BSA-FITC solutions in phosphate buffer saline solution (PBS) at pH 7 at a concentration of 2 mg mL⁻¹ and incubated for 1 h in the dark to form BSA adlayers at the COP surface. Coupons were then rinsed in (method 1) PBS; or (method 2) in PBS and in elution buffer 1 (EB1=PBS+1% Triton X), as schematically depicted in FIG. 3b. Method 1 is expected to leave the largest amount of protein adsorbed, consisting of both soft and hard adsorbed layers of BSA. Method 2 is expected to remove most of the soft layer. After rinsing via methods 1&2, the adhered BSA-FITC was extracted into a 1 mL volume for quantitation via fluorescence methods. The extraction protocol consisted of incubation for 17 h in EB1 with addition of mercaptoethanol at 1% as a proteolytic agent, in order to fragment the protein and quantitatively release the FITC label into solution. The emission intensity from the extracted solution at 495 nm excitation was used to quantitate the protein via calibration with BSA-FITC standards.

The present study shows the effects of a surface modification using polysaccharides that shows significant promise in addressing protein adsorption.

Other work has shown that the protein rejection is observed also on the inner surface of syringes used for biotherapeutics, on COP materials. Protein rejection appears to be general, as it is observed with a general probe globular protein and with a therapeutic protein of smaller size.

### Examples

Surface modification protocols. The surface modification protocols used 1.25 cm² coupons of TOPAS™ (T), ZEONOR™ (Z) and ZEONEX (ZX); these were subject to two different types of pre-treatment prior to modification with saccharides (id1 # in sample nomenclature):
1) Rinsing with millipore water (TW, ZW or ZXW)
2) Mild surface oxidation using hydrogen peroxide 30% at 50° C. (TP50, ZP50 or ZXP50). The pre-treated coupons were subsequently incubated in 1 mg mL⁻¹ solutions of different saccharides to carry out modifications of the surface. Scheme 2 shows the structures of polysaccharides tested in our experiments (id2 # in sample nomenclature): dextran (D), polygalacturonic acid (PGA), hyaluronic acid (H) or no saccharide (NS). The following incubation conditions were tested (id3 # in sample nomenclature):
1) Saccharide 1 mg mL⁻¹ in deionised water at room temperature for 2 h (W) 2) Saccharide 1 mg mL⁻¹ in deionised water at 50° C.; 4 consecutive incubations of 30 min (total 2 h) (W50X4).
3) Saccharide 1 mg mL⁻¹ in 30% H₂O₂ at 50° C.; 4 consecutive incubations of 30 min (total 2 h) (P50X4).

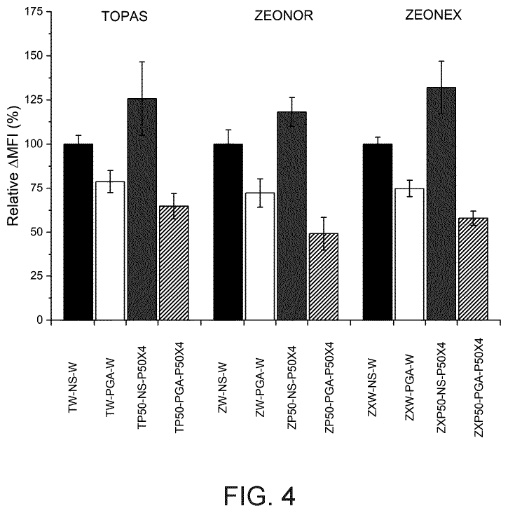

Following the incubation period, all samples were rinsed in deionised water and used for screening the amount of protein adsorption. To identify the treatment undergone by each surface tested, samples are referred by the combination of pre-treatment (id1 #), saccharide (id #2) and modification treatment (id3 #) used, as shown in FIG. 4.

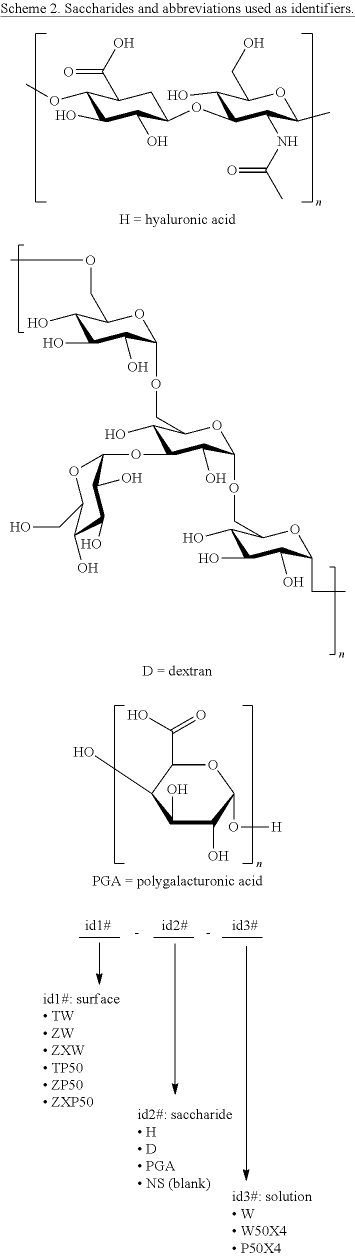

Protein adsorption testing protocol. Solutions of BSA-FITC were prepared in phosphate buffer saline solutions (PBS) at pH 7 at concentrations of 2 mg mL⁻¹. Coupons of the COP materials were immersed in BSA-FITC solutions and incubated for 1 h in the dark. The materials were then rinsed in (method 1) PBS and used for either quantitative or qualitative determinations as below:

a. Quantitative determinations via emission from solution. After rinsing the adhered BSA-FITC was extracted into a 1 mL volume for quantitation via fluorescence methods. The extraction protocol consisted of incubation for 17 h in EB1 with addition of mercaptoethanol at 1% as a proteolytic agent, in order to fragment the protein and quantitatively release the FITC label into solution. The emission intensity from the extracted solution at 470 nm excitation was used to quantitate the protein via calibration with BSA-FITC standards. The protein surface coverage was calculated by normalising the total extracted protein by the exposed COP area during incubation. Error bars in all graphs correspond to 95% C.I.

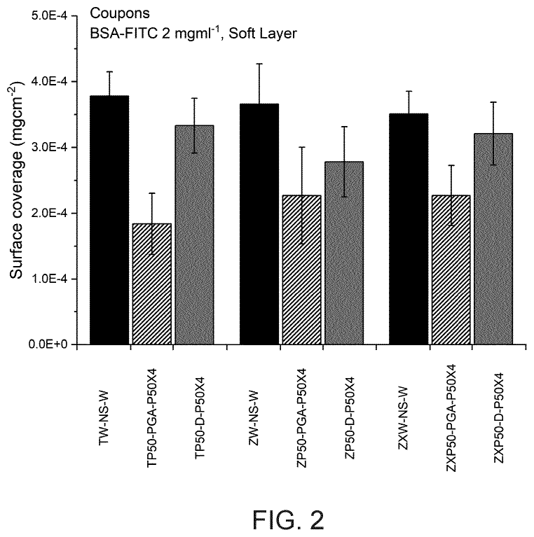

b. Qualitative comparisons via fluorescence microscopy. After rinsing the coupons were imaged using upright microscope with 470 nm excitation and a FITC exc/em filter cube to determine the integrated intensity at the COP surface via commercial software. Method 1 makes the method sensitive to both soft and hard adsorbed layers (FIG. 2). The mean fluorescence intensity (MFI) through the emission filter was measured from multiple images and corrected by the background emission (ΔMFI) of the corresponding pristine COP material. Error bars in all graphs correspond to 95% C.I.

BSA-FITC Adsorption Results on COP Coupons

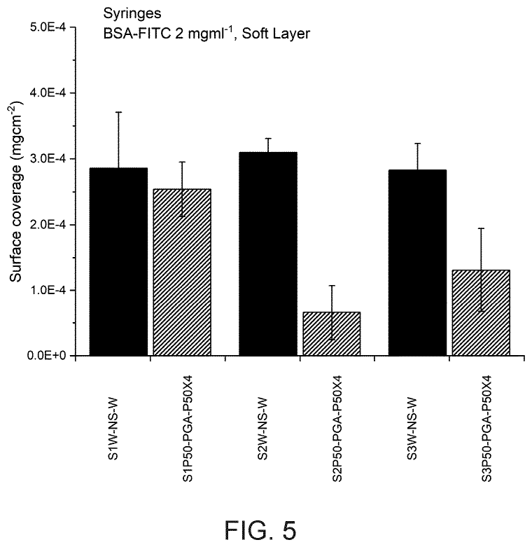

FIG. 5 shows results from quantitative determinations of BSA-FITC adsorption at Topas™, Zeonor™ and Zeonex surfaces. The ##-NS-W samples provide controls as it mimics the expected adsorption at e.g. a syringe barrel without any pre-treatment or modification. It is clear that modification with PGA polysaccharides yield the best reductions in the density of protein adsorbates. The best reduction is of 52% and observed for TP50-PGA-P50X4. Table 1 shows a summary of protein rejection results calculated as adsorption relative to the pristine coupon surfaces.

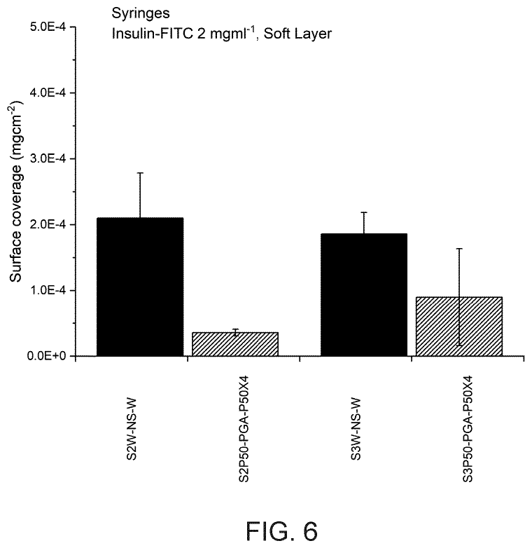

Protein adsorption changes were also confirmed via qualitative fluorescence microscopy methods as shown in FIG. 6. Emission from the coupon surface detected via microscopy shows that PGA-treatment results in lower emission from adsorbed BSA-FITC on all types of COP coupons tested.

TABLE 1
Summary of results of protein rejection measurements
calculated from average values shown in FIG. 5.

| | TOPAS ™ | ZEONOR ™ | ZEONEX ™ |
|---|---|---|---|
| Polygalacturonic acid | 52% | 38% | 35% |
| Dextran | 13% | 24% | 7% |
| Hyaluronic acid | — | 8% | — |

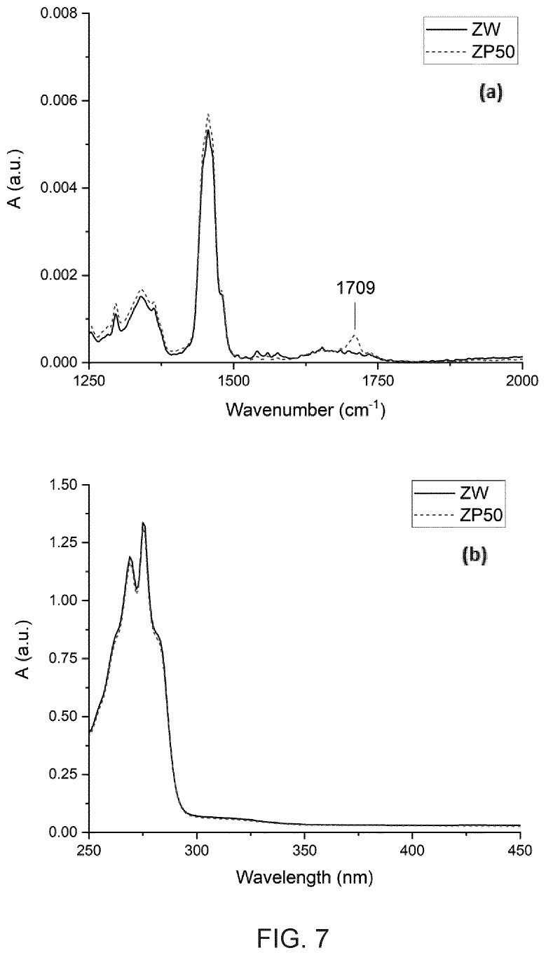

FIG. 7 shows the total emission from adsorbed BSA-FITC on the three polymer materials tested after the coupons were treated with PGA alone, with hydrogen peroxide alone or using the combination of PGA and peroxide treatment. It is evident that PGA alone does not result in as significant a reduction as when the surface is also treated with peroxide; whereas peroxide has a largely negative effect on protein rejection unless PGA is added to the treatment solutions.

Protein Adsorption Results on COP Syringes

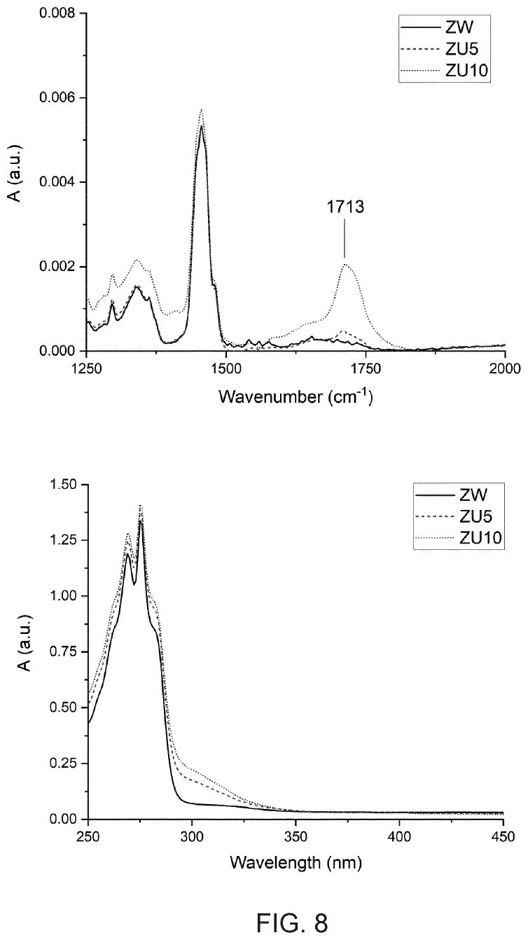

FIG. 8 shows results from quantitative determinations of BSA-FITC adsorption at Manufacturer #1, #2 and #3 COP syringes. The ##-NS-W syringes provide controls as they report the expected adsorption at clean syringe barrels without any pre-treatment or modification. It is clear that whereas pristine syringes display surface coverage of adsorbates that is comparable to that determined on coupon samples, the PGA modifications result in a significant reduction of BSA-FITC adsorption for #1 (79%) and #2 (54%) syringes. #1 syringes do not show significant reduction. However, this is consistent with these devices being siliconized over their inner surface and therefore indicate that COP surfaces are most affected by the polysaccharide treatment directly on their surface without a silica coating. Given the success of the modification protocol on #2 and #3 syringes we expanded the quantitative determinations to a different type of protein, Insulin-FITC, a protein that in its unlabelled form is used for therapeutic applications. FIG. 6 shows results from quantitative determinations with insulin-FITC; it is clear that also with this protein the PGA modification results in a decrease on #2 (83%) and #3 (52%) syringes.

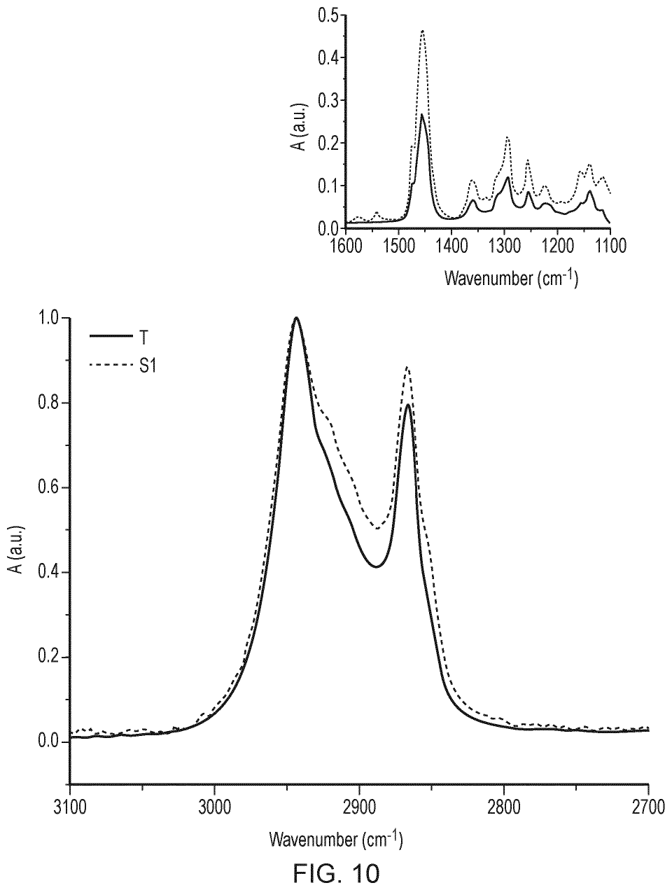

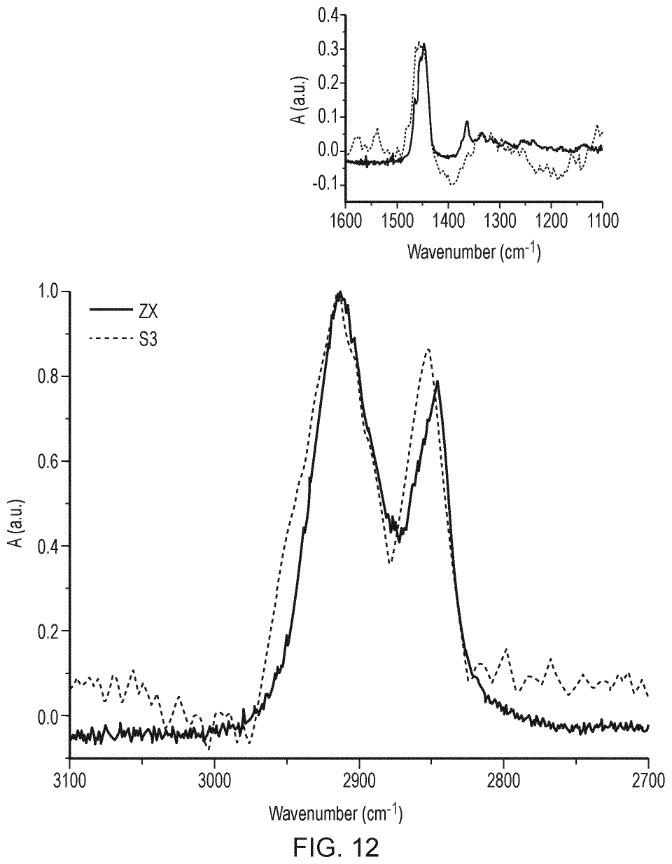

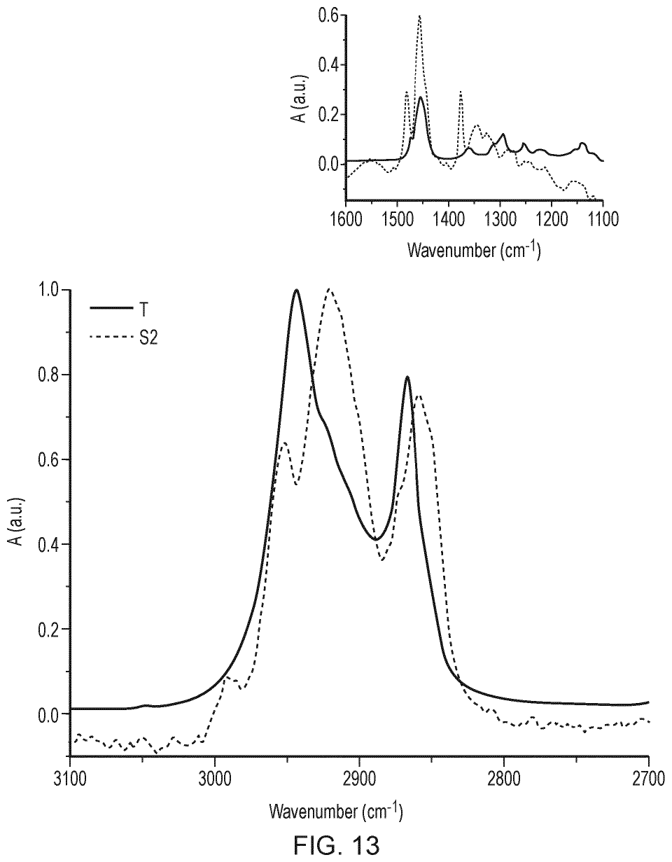

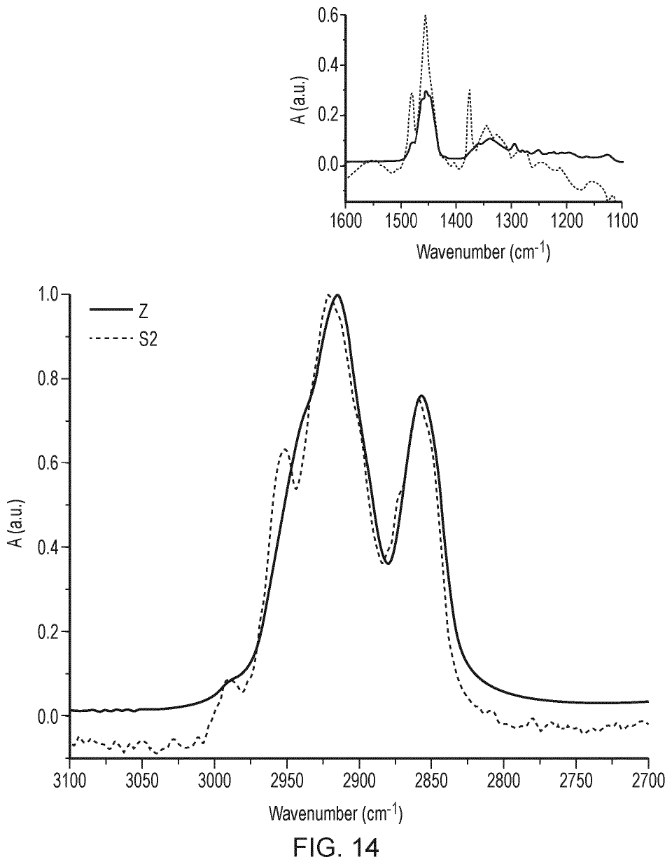

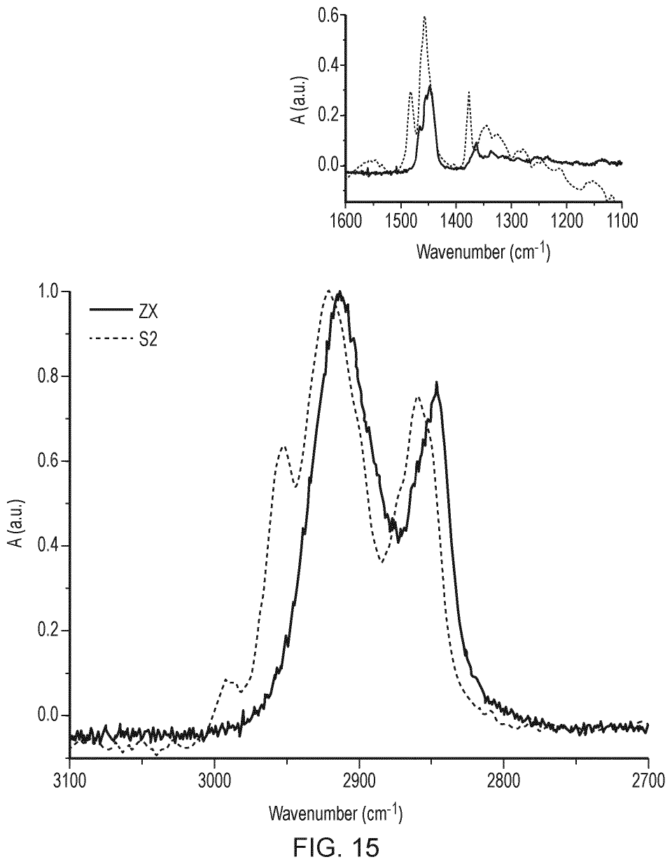

FIGS. 10 to 15 shows comparisons between the FT-IR spectra of COP materials (as coupons) and the syringe materials (types S1, S2, S3 from manufacturers #1, #2 and #3 respectively) discussed herein.

Effect of Surface Treatments on COP Materials

The effect of solution treatments and reactions conditions were investigated using Ge-attenuated total internal reflectance infrared spectroscopy (GATR-FTIR), water contact angle (WCA), and transmittance UV-Vis spectroscopy. FIG. 10a shows GATR-FTIR spectra of a COP coupon before and after exposure to H₂O₂ at 50° C.; the spectra show the appearance of a clear absorbance peak at 1709 cm⁻¹ that is diagnostic of carbonyl functional groups. This indicates that exposure to peroxide at the reaction conditions results in oxidative activation of the COP. This oxidation is however mild and confined to the surface of the material as shown by control UV-Vis absorbance spectra in FIG. 10b, that indicates no change in the bulk optical properties.

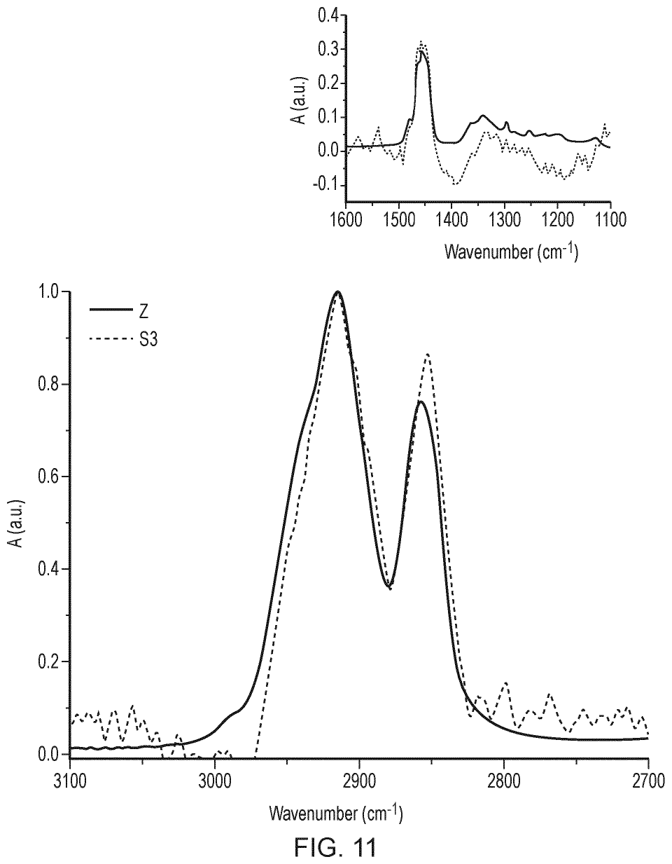

This is to be contrasted with other methods of surface oxidation such as exposure to UV/ozone lamp; this is shown in FIGS. 11a and 11b where the GATR and UV-Vis absorbances of the same type of COP coupon are shown after oxidation via UV/ozone lamp irradiation (10 min). Although the appearance of carbonyl peaks is apparent in the GATR-FTIR spectrum after oxidation, there is a significant increase in the optical absorbance in the UV-Vis absorbance spectrum, that is diagnostic of changes to the bulk structure of the COP polymer. The oxidation with H₂O₂ is therefore relatively mild and does not significantly alter the bulk material.

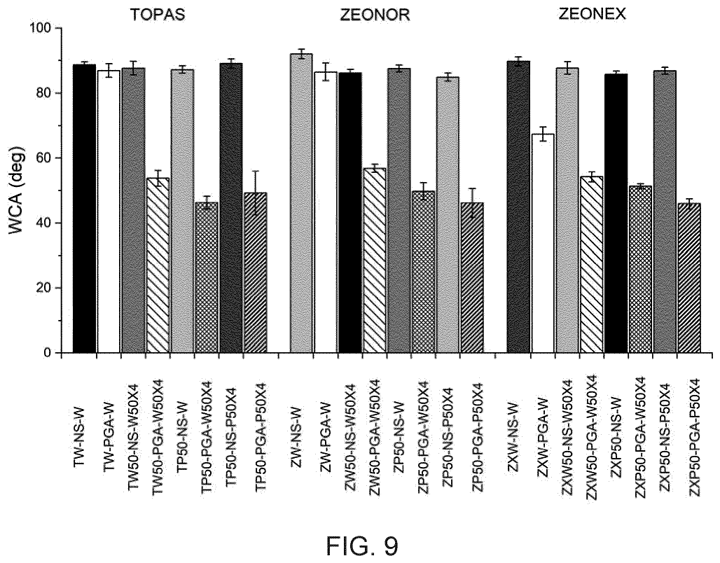

WCA measurements were used to monitor changes in hydrophilic character resulting from the surface treatments. FIG. 9 shows WCA values obtained at COP surfaces of the three polymers treated with and without PGA under different conditions. Results indicate that after H₂O₂ exposure alone only slight changes in hydrophilic character are observed; however, exposure to PGA results in significant increases in hydrophilic character.

### CONCLUSION

For COP materials, a process of surface oxidation in combination with immobilization of a polysaccharide reduces still further protein adsorbates.

Protein rejection appears to be general, as it is observed with a general probe globular protein and with a therapeutic protein of smaller size.

The disclosures of the published documents referred to herein are incorporated by reference in their entirety.

1.-25. (canceled)

26. A method of coating a polymer surface, the method comprising:
a) providing a polymer having a surface,
b) optionally, treating at least a portion of the polymer surface with an oxidising agent,
c) treating at least a portion of the polymer surface with a composition comprising a polysaccharide, oligosaccharide, polyol or mixture thereof, and
d) incubating the treated polymer with the composition for a predetermined time to form a coated polymer.

27. The method of claim 26, wherein the polymer comprises a cyclic olefin polymer and/or co-polymer.

28. The method of claim 26, wherein the polysaccharide comprises a hexose derived polysaccharide or oligosaccharide.

29. The method of claim 26, wherein the polysaccharide comprises 20 percent or greater oxidised hexose at the C6 position.

30. The method of claim 26, wherein the polysaccharide is selected from dextrin, dextran polygalacturonic acid, hyaluronic acid, or a combination of two or more of these polysaccharide.

31. The method of claim 26, wherein the oxidising agent comprises a peroxide, optionally comprises hydrogen peroxide, optionally hydrogen peroxide in 30% w/w aqueous solution.

32. The method of claim 26, wherein the predetermined time is in the range of 0.5 mins to 240 mins.

33. The method of claim 26, wherein the step of treating at least a portion of the polymer surface and/or the step of incubating is at a temperature in the range 10° C. to 90° C.

34. The method of claim 26, wherein the composition comprises water.

35. The method of claim 26, wherein the composition comprises an oxidising agent.

36. The method of claim 35, wherein the oxidising agent in the composition comprises a peroxide, O₃, ozonated water, H₂O₂, periodate, hypochlorite, and/or permanganate, optionally comprises hydrogen peroxide, optionally comprises hydrogen peroxide in 30 percent w/w aqueous solution.

37. The method of claim 26, further comprising the step of forming a vessel comprising the coated polymer.

38. The method of claim 37, wherein the vessel is selected from the group consisting of a multi-well plate, a pipette, a bottle, a flask, a vial, an Eppendorf tube, and a culture plate.

39. The method of claim 37, further comprising the step of storing a pharmaceutical proteinaceous composition in the vessel.

40. The method of claim 26, further comprising the step of forming a medical device comprising the coated polymer, wherein the medical device is selected from the group consisting of a dispensing tube, a device comprising a channel, and a syringe.

41. A method of reducing protein or oligonucleotide aggregation and/or adsorption on a polymer surface, the method comprising,
a) providing a polymer having a surface coating, the surface coating comprising a polysaccharide oligosaccharide, polyol or mixture thereof directly contacting the surface of the polymer; and
b) contacting the surface coating with a proteinaceous composition or a composition comprising an oligonucleotide.

42. The method of claim 41, wherein the proteinaceous composition comprises a pharmaceutical proteinaceous composition comprising a monoclonal antibody composition, or a peptide hormone.

43. A polymer having a coating on at least one surface, the coating comprising a polysaccharide oligosaccharide, polyol or mixture thereof directly contacting the surface of the polymer.

44. The polymer of claim 43, wherein the polymer comprises a cyclic olefin polymer.

45. The polymer of claim 43, wherein the polysaccharide comprises dextrin, polygalacturonic acid, hyaluronic acid, or a combination of two or more of these polysaccharides.
